(19) United States
(12) Patent Application Publication
(10) Pub. No.: US 2026/0020985 A1
(43) Pub. Date: Jan. 22, 2026

(54) METHOD FOR PROVIDING CONTROL DATA FOR AN OPHTHALMOLOGICAL LASER OF A TREATMENT APPARATUS, CONTROL DEVICE, TREATMENT APPARATUS, COMPUTER PROGRAM AS WELL AS COMPUTER-READABLE MEDIUM

(71) Applicant: SCHWIND EYE-TECH-SOLUTIONS GMBH, Kleinostheim (DE)

(72) Inventors: Samuel ARBA MOSQUERA, Aschaffenburg (DE); Nico TRIEFENBACH, Mainaschaff (DE)

(21) Appl. No.: 19/269,591

(22) Filed: Jul. 15, 2025

(30) Foreign Application Priority Data
Jul. 18, 2024 (DE) 10 2024 120 358.7

(51) Int. Cl. A61F 9/008

(52) U.S. Cl. CPC A61F 9/00814; A61F 9/0084; A61F 2009/00872; A61F 2009/00882; A61F 2009/00897

(57) ABSTRACT

The invention relates to a method for providing control data for an ophthalmological laser (12) of a treatment apparatus (10) as well as to an surgical procedure, wherein the method comprises the following steps performed by at least one control device (18): Presetting a course of a Bowman's membrane (34) of a cornea of an eye (14) of a patient depending on at least one patient information; determining an incision course at least for an anterior interface (20) of a volume body (16) to be removed in a stroma (36) of the eye (14) depending on the course of the Bowman's membrane (34) such that a preset geometric relation to the Bowman's membrane (34) is formed by the anterior interface (20); and generating the control data for controlling the laser (12) by means of the control device (18) such that it emits pulsed laser pulses in a shot sequence onto the eye. Further, the invention relates to a control device (18), to a treatment apparatus (10), to a computer program as well as to a computer-readable medium.

### FIELD

The following invention relates to a method for providing control data for an ophthalmological laser of a treatment apparatus as well as to a method for performing a surgical procedure, wherein the method is performed by a control device. Further, the invention relates to a control device, to a treatment apparatus, to a computer program as well as to a computer-readable medium.

### BACKGROUND

Opacities and scars within the cornea, which is also referred to as cornea, which can arise by inflammations, injuries or native diseases, impair the vision. In particular in case that these pathological and/or unnaturally altered areas of the cornea are situated in the axis of vision of the eye, clear sight is considerably disturbed. Furthermore, further vision defects, such as for example a reduced visual acuity or corneal curvatures, can also impair the vision. Hereto, different laser methods by means of corresponding treatment apparatuses are given from the prior art, which separate a volume body from the cornea and thus can improve the sight for a patient. Hereto, photodisruptive and ablative methods are for example known, which generate corresponding interfaces via laser pulses, and thereby a volume body can for example be removed from the cornea, whereby the injured or pathological area can be changed such that the sight is again improved.

### SUMMARY

It is the object of the present invention to provide a method, a control device, a treatment apparatus, a computer program as well as a computer-readable medium, by means of which improved control data can be generated for an ophthalmological laser of a treatment apparatus.

This object is solved by the method according to the invention, the apparatuses according to the invention, the computer program according to the invention as well as the computer-readable medium according to the invention. Advantageous configurations with convenient developments of the invention are presented in the respective dependent claims and/or other embodiments disclosed herein, wherein advantageous configurations of the method are to be regarded as advantageous configurations of the treatment apparatus, of the control device, of the computer program and of the computer-readable medium and vice versa.

A first aspect of the invention relates to a method for providing control data for an ophthalmological laser of a treatment apparatus, wherein the method is performed by one or more control devices. A further aspect relates to a corresponding method for performing a surgical procedure on an eye. A course of a Bowman's membrane of a cornea of an eye of a patient is preset depending on at least one patient information. Determining an incision course at least for an anterior interface of a volume body to be removed in a stroma of the eye depending on the course of the Bowman's membrane is effected such that a preset geometric relation to the Bowman's membrane is formed by the anterior interface. Control data for controlling the laser is generated by means of the control device such that it emits pulsed laser pulses to the eye for performing a photoablation and/or for performing a photodisruption.

In particular, this has the advantage that an incision can be correspondingly generated depending on a course of the Bowman's membrane, which in particular prevents the Bowman's membrane and for example also a corresponding epithelium from being able to be impaired or injured.

Thus, an incision course can be reliably generated, in particular for the anterior interface, which correspondingly considers the course of the Bowman's membrane and thus prevents the Bowman's membrane from being injured. Thereby, a volume body can be generated in improved manner, whereby a treatment on an eye can in particular be improved based on the generated control data.

Therein, the treatment apparatus can comprise the one control device, which performs the corresponding method steps. Alternatively, multiple control devices can also be provided, which can correspondingly perform individual method steps or multiple method steps.

For example, the patient information can be correspondingly provided based on previously performed examinations. Therein, the patient information in particular includes at least one information with respect to the course of the Bowman's membrane. Furthermore, further information can also be taken from the patient information, for example information with respect to a severity of the visual disorder of the patient or a location of an opacity. For example, the patient information can be performed directly before the actual generation of the control data or the treatment. Alternatively or additionally, it is also possible that the patient information can be generated in an examination of the patient occurring before the treatment.

In particular, it can then be provided that the control data is generated such that it correspondingly controls the laser, in particular controls it in automated manner, such that an intervention by a further person is substantially not required.

Therein, the invention further in particular proposes that in case of an unwanted misalignment of a patient interface during a treatment, it is also prevented in the outer area of the incision that the Bowman's membrane is correspondingly injured.

Therein, the method can be applied in an ablation method, in which a so-called flap is for example generated by means of photodisruption and a treatment by means of ablation is performed after generation of the flap. Alternatively, the laser pulses can be generated for a treatment by means of photodisruption. Herein, the laser pulses are emitted into the cornea, and a plurality of cavitation bubbles arises by interaction with the cornea, by means of which at least one interface of a volume body can then in turn be generated, which can be removed from the cornea.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

According to an advantageous configuration, a preset distance to the Bowman's membrane is complied with as the preset geometric relation. In other words, it is provided that a distance, which for example has been previously preset by a person, is correspondingly complied with. Thus, the anterior interface is generated such that it complies with the preset distance to the Bowman's membrane. Therein, the distances in a center of the anterior interface can for example be different than at an outer edge of the anterior interface. In other words, multiple distances can be preset, within which the incision course for the anterior interface in relation to the Bowman's membrane is in turn located. Thus, the Bowman's membrane can be prevented from being correspondingly impaired, wherein it can be additionally prevented by the different distances that an impairment of the Bowman's membrane can be registered even upon misalignment of a patient interface.

Further, it has proven to be advantageous if a course of an epithelial layer of the eye is additionally considered in determining the incision course. Thus, the injury of an epithelium can in particular also be correspondingly prevented. This allows an improved treatment for a patient, since the course of the epithelial layer is now also considered.

A further advantageous form of configuration provides that the incision course is determined such that the incision course has a different curvature than a curvature/course of the Bowman's membrane. In particular, the distance to the Bowman's membrane can for example be increased at the outer edges of the anterior interface with respect to a center of the anterior interface. Thus, it can be prevented that an injury or impairment of the Bowman's membrane can be registered even in case of a misalignment of the patient interface. Thus, an improved treatment can be realized by the corresponding control data.

Therein, it has further proven to be advantageous if the curvature of the incision course is determined greater than the curvature of the Bowman's membrane. Thus, it is in particular provided that a distance between Bowman's membrane and the anterior interface is larger at the outer edges than in the center of the incision course such that, as already mentioned, an injury of the Bowman's membrane can be prevented even in case of a misalignment of the patient interface.

It is also advantageous if the curvature of the incision course is determined equal to a curvature of the Bowman's membrane. Thus, the curvature of the anterior interface can be determined in simple manner, and an injury of the Bowman's membrane is prevented at the same time. Thus, the generation of the control data can be performed reduced in effort.

At this point, it is to be pointed out that it can for example also be provided that the incision course is determined such that the incision extends substantially parallel to the Bowman's membrane for example on a first side of the volume body, thus a same distance between the anterior interface and the Bowman's membrane can be registered, and a larger distance and thus a greater curvature of the incision course is present on a second side of the incision course opposing the first side. Thus, the incision course can be correspondingly determined specific to patient. Furthermore, already performed treatments can for example also be taken into consideration.

It has further proven to be advantageous if the laser pulses are emitted in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz. Thus, a treatment can be reliably realized by means of the laser.

In a further advantageous form of configuration, topographic and/or pachymetric and/or morphologic data of the optical element, in particular of the eye, in particular of the cornea and/or the lens, is considered in controlling the laser. In particular, this data can for example be determined already before a treatment. Based on this data, the treatment can then be reliably performed.

A further aspect of the invention relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one of the previously described methods. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be encompassed by a computer or computer cluster.

A still further aspect of the invention relates to a treatment apparatus with at least one eye surgical laser and with at least one control device for the laser or lasers, which is formed to perform the steps of the method according to the first or the second aspect.

Preferably, the treatment apparatus is formed as a rotation scanner and hereto for example comprises a beam deflection device.

In an advantageous form of configuration of the treatment apparatus, the treatment apparatus comprises a storage device for at least temporary storage of at least one control dataset, wherein the control dataset or datasets include(s) control data of individual laser pulses on or in the optical element, and includes at least one beam deflection device for beam guidance and/or beam shaping and/or beam deflection and/or beam focusing of a laser beam of the laser. Therein, the mentioned control datasets are usually generated based on a measured topography and/or pachymetry and/or morphology of the optical element to be treated, in particular the cornea or lens to be treated, of the pathologically and/or unnaturally altered area within the optical element.

Therein, it can be provided that the treatment apparatus comprises a single storage device and a single control device. Alternatively, it can be provided that different storage devices and control devices are formed within the treatment apparatus, to perform a corresponding control of the laser. The respective laser can be formed to at least partially separate a predefined corneal volume with predefined interfaces of a human or animal eye by means of optical breakdown, in particular at least partially separate it by means of photodisruption and/or to ablate corneal layers by means of (photo)ablation.

A further aspect of the invention relates to a computer program. The computer program includes commands, which for example form a program code. The program code can include at least one control dataset with the respective control data for the respective laser. Upon execution of the program code by means of a computer or a computer cluster, it is caused to execute the previously described method or at least one embodiment thereof.

A still further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the developments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 a schematic block diagram according to an embodiment of a treatment apparatus with an embodiment of a control device;

FIG. 2 a schematic sectional image of an eye with a first incision course made based on control data according to an embodiment of the method; and

FIG. 3 a further schematic sectional view of an eye with a second incision course made based on control data according to a further embodiment of the method.

In the figures, identical or functionally identical elements are provided with the same reference characters.

### DETAILED DESCRIPTION

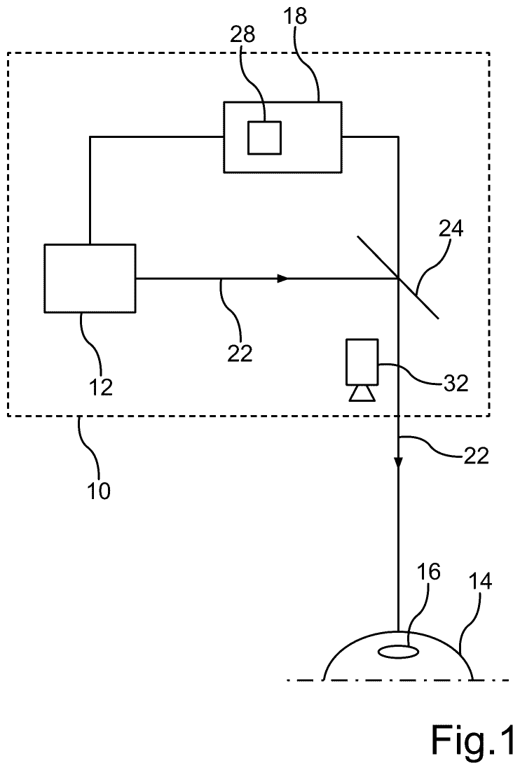

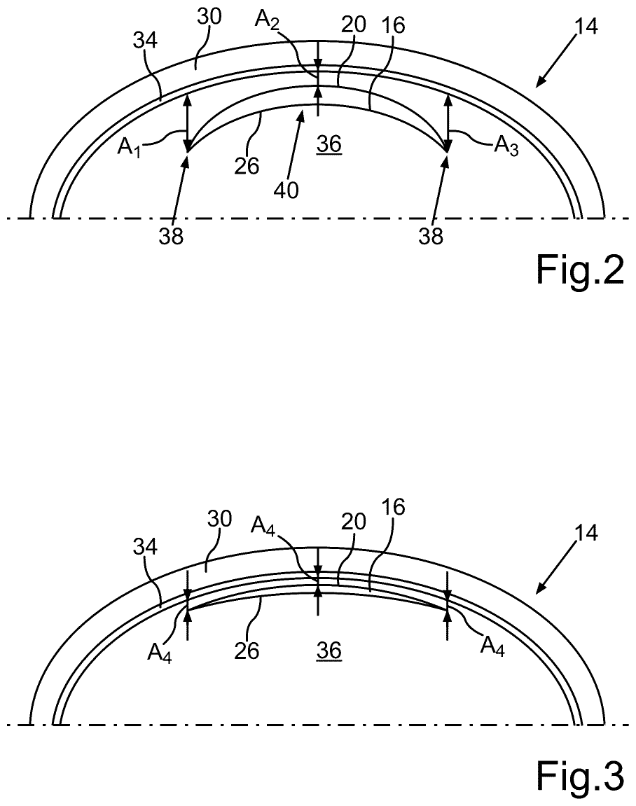

FIG. 1 shows a schematic representation of a laser apparatus 10 with a laser 12 for example for the treatment of a patient, in particular for the treatment of an eye 14 of a patient, wherein the eye 14 can also be referred to as optical element below. Thus, the laser apparatus 10 is presently formed as an eye surgical/ophthalmological treatment apparatus. One recognizes, that a control device 18 for the laser 12 is also formed besides the laser 12. This form of configuration with a control device 18 is to be purely exemplarily regarded. It can be provided that the laser apparatus 10 also comprises a plurality of, in particular more than two, control devices 18. For example, the control device 18 can emit pulsed laser pulses in a predefined pattern into the eye 14, for example into an area, wherein the position of the area is selected in this embodiment such that a pathological and/or unnaturally altered area within a stroma 36 (FIG. 2) of the eye 14 is enclosed. Thus, the area is an area to be treated.

Furthermore, one recognizes that the laser beam 22 generated by the laser 12 is deflected towards the eye 14 by means of a beam deflection device 24, such as for example a scanner, in particular a so-called rotation scanner. The beam deflection device 24 is also controlled by the control device 18, to for example generate irradiation lines. For example, the beam deflection device 24 can comprise one or also two mirrors, which are formed for deflecting the impinging laser beam 22.

In the present embodiment, the illustrated laser 12 is a laser 12, which emits the laser pulses in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

In addition, the control device 18 comprises a storage device 28 for at least temporary storage of at least one control dataset, wherein the control dataset or datasets include(s) control data for positioning and/or for focusing individual laser pulses in or on the eye 14. The position data and/or focusing data of the individual laser pulses is generated based on a previously measured topography and/or pachymetry and/or the morphology of the eye 14 and the for example pathological and/or unnaturally altered area within the stroma of the eye 14. Furthermore, a capturing device 32, for example in the form of a camera, is shown, by means of which monitoring of a treatment can be performed.

FIG. 2 shows a schematic sectional view of an eye 14 with a volume body 16. In the present embodiment, the volume body 16 comprises an anterior interface 20 as well as a posterior interface 26. In the present embodiment, in other words, the volume body 16, which could be generated by controlling the laser 12 with the corresponding control data, is already formed. In the present embodiment, the eye 14 in particular comprises an epithelial layer 30 as well as a Bowman's membrane 34.

In the present embodiment, the volume body 16 can in particular be generated by means of photodisruption. Hereto, the laser pulses are in particular emitted into the cornea, such that a plurality of cavitation bubbles arises with interaction with the cornea, by which the interfaces 20, 26 can be generated. Alternatively, an ablation method can also be applied.

According to an embodiment of the method, it is provided that a course of the Bowman's membrane 34 of a cornea of the eye 14 of a patient is preset depending on at least one patient information. An incision course at least for the anterior interface 20 of the volume body 16 to be removed in the stroma 36 of the eye 14 is determined depending on the course of the Bowman's membrane 34 such that a present geometric relation A₁-A₄ is formed by the anterior interface 20. Then, generating the control data for controlling the laser 12 by means of the control device 18 is effected such that it emits pulsed laser pulses in a shot sequence into the cornea, wherein at least the anterior interface 20 is generated by means of an interaction of the individual laser pulses with the cornea by the generation of a plurality of cavitation bubbles according to this embodiment. For example, the posterior interface 26 can also be generated via a plurality of cavitation bubbles.

In particular, it is provided that a preset distance A₁-A₄ to the Bowman's membrane 34 is complied with as the preset geometric relation. Furthermore, it can be provided that a course of the epithelial layer 30 of the eye 14 is additionally considered in determining the incision course.

Therein, FIG. 2 in particular shows that the incision course can be determined such that the incision course has a different curvature than the course of the Bowman's membrane 34. Herein, it is in particular shown in the present embodiment that a first distance A₁ at an outer area 38 of the volume body 16 is larger than in a central area 40 of the volume body 16. In other words, a second distance A₂ to the Bowman's membrane 34 at the center is lower than the first distance A₁ in the present embodiment. Furthermore, it is shown that a third distance A₃ is also correspondingly larger than the second distance A₂. Therein, the first distance A₁ and the third distance A₃ can be formed identical or also different in the present embodiment. Thus, FIG. 2 in particular shows that the curvature of the incision course is determined greater than the curvature of the Bowman's membrane 34.

FIG. 3 again shows that the curvature of the incision course is formed equal to the curvature of the Bowman's membrane 34. In other words, the anterior interface 20 always has a same distance to the Bowman's membrane 34, which is presently shown as a fourth one A₄.

Thus, it is in particular shown that the volume body 16 can be formed both as a cap and as a flap, wherein it is for example generated parallel to the Bowman's membrane 34, in particular at least the anterior interface 20, as illustrated in FIG. 3. Thereby, it can for example be prevented that an impairment of the Bowman's membrane 34 can be registered even in case of a misaligned arrangement of the patient interface itself. Thus, a misalignment mitigation measure is in particular effected by overbending the anterior interface 20 with respect to the Bowman's membrane 34. In particular with correspondingly curved contact elements or patient interfaces, this has the advantage that the constant distance between the anterior interface 20 and the patient interface does not always have to be complied with, but that different distances between the patient interface and the anterior interface 20 can also be formed. Thus, it can in particular be prevented that too thin stromal incisions are performed, which can result in the transection of the Bowman's membrane 34, up to an injury of the epithelial layer 30.

1. A method for providing control data for an ophthalmological laser of a treatment apparatus, wherein the method comprises the following steps performed by at least one control device:
presetting a course of a Bowman's membrane of a cornea of an eye of a patient depending on at least one patient information;
determining an incision course at least of an anterior interface of a volume body to be removed in a stroma of the eye depending on the course of the Bowman's membrane such that a preset geometric relation to the Bowman's membrane is formed by the anterior interface; and
generating the control data for controlling the laser by means of the at least one control device such that it emits pulsed laser pulses in a shot sequence onto the eye for performing a photoablation and/or for performing a photodisruption for generating the volume body.

2. The method according to claim 1, wherein
a preset distance to the Bowman's membrane is complied with as the preset geometric relation.

3. The method according to claim 1, wherein
a course of an epithelial layer of the eye is additionally considered in determining the incision course.

4. The method according to claim 1, wherein
the incision course is determined such that the incision course has a different curvature than a curvature of the Bowman's membrane.

5. The method according to claim 4, wherein
the curvature of the incision course is determined greater than the curvature of the Bowman's membrane.

6. The method according to claim 1, wherein
a curvature of the incision course is determined equal to the curvature of the Bowman's membrane.

7. The method according to claim 1, wherein
the laser is provided such that it emits laser pulses in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

8. The method according to claim 1, wherein
control of the laser is effected such that topographic and/or pachymetric and/or morphologic data of the cornea is considered.

9. The method according to claim 1, further including the step of
transferring the control data to a respective ophthalmological laser of the treatment apparatus.

10. A control device, which is configured to perform a respective method according to claim 1.

11. A treatment apparatus with at least one ophthalmological laser for the separation of a corneal volume as a volume body with predefined interfaces of a human or animal eye by means of optical breakdown, in particular by means of photodisruption and/or photoablation, and at least one control device according to claim 10.

12. A non-transitory computer-readable medium having a computer program stored thereon, the computer program including commands, which cause a treatment apparatus to execute the method according to claim 1.

13. (canceled)

14. A method for performing a surgical procedure on an eye by an ophthalmological laser of a treatment apparatus, wherein the method comprises the following steps performed by at least one control device:
presetting a course of a Bowman's membrane of a cornea of the eye of a patient depending on at least one patient information;
determining an incision course at least of an anterior interface of a volume body to be removed in a stroma of the eye depending on the course of the Bowman's membrane such that a preset geometric relation to the Bowman's membrane is formed by the anterior interface; and
generating control data for controlling the laser by means of the at least one control device such that it emits pulsed laser pulses in a shot sequence onto the eye for performing a photoablation and/or for performing a photodisruption for generating the volume body.

15. The method according to claim 14, wherein
a preset distance to the Bowman's membrane is complied with as the preset geometric relation.

16. The method according to claim 14, wherein
a course of an epithelial layer of the eye is additionally considered in determining the incision course.

17. The method according to claim 14, wherein
the incision course is determined such that the incision course has a different curvature than a curvature of the Bowman's membrane.

18. The method according to claim 17, wherein
the curvature of the incision course is determined greater than the curvature of the Bowman's membrane.

19. The method according to claim 14, wherein
a curvature of the incision course is determined equal to the curvature of the Bowman's membrane.

20. The method according to claim 14, wherein
the laser is provided such that it emits laser pulses in a wavelength range between 200 nm and 2 μm, in particular between 400 nm and 1450 nm, at a respective pulse duration between 1 fs and 1 ps, in particular between 10 fs and 100 fs, and a repetition frequency of greater than 10 kHz, in particular between 1 MHz and 100 MHz.

21. The method according to claim 14, wherein
the control of the laser is effected such that topographic and/or pachymetric and/or morphologic data of a cornea is considered.
